(19) United States
(12) Patent Application Publication
(10) Pub. No.: US 2020/0359958 A1
(43) Pub. Date: Nov. 19, 2020

(54) SLEEP SUFFICIENCY ESTIMATION DEVICE AND SLEEP SUFFICIENCY ESTIMATION METHOD

(71) Applicant: National Institute of Advanced Industrial Science and Technology, Tokyo (JP)

(72) Inventor: Kosuke Kaida, Tsukuba-shi (JP)

(21) Appl. No.: 16/945,018

(22) Filed: Jul. 31, 2020

Related U.S. Application Data
(63) Continuation-in-part of application No. PCT/JP2018/044758, filed on Dec. 5, 2018.

(30) Foreign Application Priority Data
Feb. 2, 2018 (JP) 2018-017491

(51) Int. Cl. A61B 5/00; G16H 50/20; G16H 50/30

(52) U.S. Cl. CPC A61B 5/4815; G16H 50/30; G16H 50/20

(57) ABSTRACT

An initial level of the sleep sufficiency levels is determined from the maximum sleep duration and an ordinary sleep duration of the person to be examined, evaluation on the ordinary sleep duration of the person to be examined himself/herself, or data obtained from persons of her than person to be examined. The sleep sufficiency level estimation section calculates an increase/decrease of the sleep sufficiency level corresponding to the sleep duration of the person to be examined by using a function selected according to characteristic data indicating characteristics of the person to be examined obtained from the person to be examined and the sleep duration zone to which the sleep sufficiency level of the person to be examined belongs. The sleep sufficiency level estimation section estimates the sleep sufficiency level of the person to be examined by using the calculated result.

### BACKGROUND OF THE INVENTION

### Field of the Invention

The present invention relates to a method for estimating sleep sufficiency and a sleep sufficiency estimation system for estimating a state of sleep sufficiency.

### Description of Related Art

Conventional arts for evaluating a sleep state of a person are disclosed or proposed in JP-A-2003-216734 (Patent Literature 1) and JP-A-2017-164580 (Patent Literature 2).

However, the conventional arts disclosed in Patent Literature 1 and Patent Literature 2 can only evaluate good or bad of the sleep state by converting the sleep state into scores based on answers or data obtained from a person to be examined. Therefore, there is a problem that it is difficult that the person to be examined knows a sleep sufficiency level of himself/herself. The “sleep sufficiency level” means a level indicating to what degree a sleep duration has been acquired with respect to the maximum value of the sleep duration necessary for the person to be examined. If the person to be examined can know the sleep efficiency level of this time after sleep, the followings can be found. First, it is found whether the sleep of this time is sufficient or insufficient as compared with sleep in the past by comparing the sleep sufficiency level of this time with sleep sufficiency levels obtained by the person to be examined from sleep in the past. It is also found whether the person to be examined is in a state where a necessary sleep duration is not acquired, in a state where the necessary sleep duration is acquired, or whether an excessive sleep duration is acquired. However, it has been difficult to estimate the sleep sufficiency level in conventional methods.

### SUMMARY OF THE INVENTION

An object of the present invention is to provide a method for estimating sleep sufficiency and a sleep sufficiency estimation system capable of estimating not only good or bad of the sleep state of a person to be examined but also the sleep sufficiency level of the person to be examined.

In the method for estimating sleep sufficiency according to the present invention, the sleep sufficiency level of the person to be examined is estimated by a first step to a fourth step.

In the first step, a plurality of functions indicating relationships between a plurality of sleep sufficiency levels and a plurality of sleep duration zones defined based on a plurality of sleep durations are prepared in consideration of sleep ecology data of a person to be examined indicating ecology relating to sleep of the person to be examined or sleep ecology data of other persons other than the person to be examined. Here, the sleep ecology data of the person to be examined indicates characteristics of ecology relating to sleep of the person to be examined, which is the data of the person to be examined such as sex, age, sleep lifestyle habits, the maximum sleep duration, an ordinary sleep duration (the sleep duration at the time of starting estimation), and the presence of sleepiness in the daytime. The sleep ecology data of other persons other than the person to be examined indicates characteristics of ecology relating to sleep of other persons other than the person to be examined, which is the data of other persons other than the person to be examined such as sex, age, sleep lifestyle habits, the maximum sleep duration, the ordinary sleep duration, and the presence of sleepiness or drowsiness in the daytime.

The sleep duration zones defined based on the sleep durations are zones of sleep durations defined based on the sleep sufficiency levels between one sleep sufficiency level and another sleep sufficiency level. In respective sleep duration zones, an increase/decrease rate of the sleep sufficiency level differs. For example, if three sleep duration zones are defined, the three sleep duration zones may be defined as zones including (1) a first sleep duration zone Z1 in which the sleep sufficiency level in a state where sleep shortage and sleep longer than necessary do not occur can be obtained due to an adequate sleep duration, (2) a second sleep duration zone Z2 in which the increase/decrease rate of the sleep sufficiency level is smaller than the increase/decrease rate in the first sleep duration zone (Z1) due to a short sleep duration and sleep shortage, and (3) a third sleep duration zone Z3 in which the increase/decrease rate of the sleep sufficiency level is equal to or smaller than the increase/decrease rate in the first sleep duration zone (Z1) due to a long sleep duration and sleep longer than necessary.

Moreover, as the plural functions indicating relationships between the plural sleep duration zones and the increase/decrease rates of the sleep sufficiency levels, an n-th order (“n” is a natural number) function in which the sleep duration is an independent variable and the increase/decrease of the sleep sufficiency level is a dependent variable may be used. A coefficient of the n-th order function may be determined according to at least one of objective data taken from the person to be examined or objective data taken from other persons, subjective data taken from the person to be examined or subjective data taken from other persons, and the increase/decrease rate of the sleep sufficiency level already determined. When the function is represented by a linear function of Y=KnX, it is preferable that X is the sleep duration, Y is an increase/decrease of the sleep sufficiency level, and Kn is the coefficient indicating the increase/decrease rate of the sleep sufficiency level per an section sleep duration in each of the plurality of sleep duration zones Zn.

In the second step, an initial level of the sleep sufficiency levels is determined from the maximum sleep duration and an ordinary sleep duration obtained from the sleep ecology data of the person to be examined or evaluation on the maximum sleep duration and the ordinary sleep duration by the person to be examined himself/herself, or data relating to the maximum sleep duration and the ordinary sleep duration obtained from other persons other than the person to be examined. That is, when the function based on the sleep ecology data of the person to be examined is used, the initial level of the sleep sufficiency levels is determined from the maximum sleep duration and the ordinary sleep duration obtained from the sleep ecology data of the person to be examined or evaluation on the maximum sleep duration and the ordinary sleep duration by the person to be examined himself/herself. When the function based on the sleep ecology data of other persons is used, the initial level of the sleep sufficiency levels is determined from data relating to the maximum sleep duration and the ordinary sleep duration obtained from other persons other than the person to be examined. Here, the “initial level of the sleep sufficient levels” is a level of a starting point at the time of estimating the sleep sufficiency level by using the function. For example, in a case where the sleep sufficiency level in the first sleep duration zone Z1 is estimated, a level of a starting point of the function used in this zone is an “initial level of the sleep sufficient levels”.

In the third step, the sleep duration of the person to be examined is measured. The measurement maybe made by using a well-known sleep duration measurement device. In order to make a measurement with high accuracy, the sleep duration can be measured from an output of an electroencephalograph attached to the person to be examined. When making a measurement simple, it is possible to measure an awakening duration by using a function of detecting the awakening duration installed on a wearable watch or the like to obtain a duration other than the awakening duration as the sleep duration.

In the fourth step, the sleep sufficiency level of the person to be examined is estimated after sleep by calculating an increase/decrease of the sleep sufficiency level from the initial level of the sleep sufficiency level corresponding to the sleep duration of the person to be examined by using the function selected from the plurality of functions according to the sleep ecology data of the person to be examined obtained from the person to be examined and the sleep duration zone to which the initial level of the sleep sufficiency level of the person to be examined belongs. For example, when the sleep ecology data of the person to be examined obtained from the person to be examined is sex/age, the sleep duration zone is selected from sex/age, the sleep duration, and the initial level of the sleep sufficiency levels, and an increase/decrease value of the sleep sufficiency level with respect to the initial level of the sleep sufficiency level corresponding to the sleep duration of the person to be examined is calculated by using the function assigned to the above zone. When the sleep sufficiency level of the person to be examined estimated after sleep is notified to the person to be examined, the person to be examined may devise a method for improving a lifestyle relating sleep based on the notification result afterward.

In the method according to the present invention, the plural functions indicating relationships between the plurality of sleep duration zones defined based on the sleep durations and the sleep sufficiency levels are prepared. Therefore, when the person to be examined changes lifestyle habits relating to sleep, the increase/decrease value of the sleep sufficiency level with respect to the initial level of the sleep sufficiency level corresponding to the sleep duration of the person to be examined is calculated by using an appropriate function corresponding to the change of the lifestyle habits, thereby estimating the sleep sufficiency level of the person to be examined at any time.

It is also preferable that the plurality of functions are further prepared in consideration of different sleep qualities corresponding to the sleep ecology of the person to be examined in the first step, and that an appropriate function is further selected from the functions prepared in consideration of the sleep quality in the fourth step. The sleep qualities may be specified as two kinds of high (H) and low (L), which are estimated from at least one of characteristics in observed brain waves and measurement values such as the number of tosses and turns, the number of vibrations of a pillow, a breathing rate, heart rate variability, blood pressure variability and a perspiration amount. This makes it possible to select a more appropriate function in consideration of the sleep qualities.

When the coefficient of the n-th order function is determined, it is preferable that the objective data includes at least one of data indicating a season, temperature, body temperature, humidity, atmospheric pressure, an exercise amount, sunshine duration, weather, meal contents, a sound exposure amount, a light exposure amount, an endocrine hormone amount, and menstrual rhythm, and that the subjective data includes at least one of data indicating a physical condition or a mood. When the above information is included in objective data and the subjective data, a more appropriate function can be obtained.

A sleep sufficiency estimation system according to the present invention for achieving the method of the present invention includes a storage section, a measurement section, and a sleep sufficiency level estimation section. The storage section stores a plurality of functions indicating relationships between a plurality of sleep sufficiency levels and a plurality of sleep duration zones defined based on sleep durations in consideration of sleep ecology data of a person to be examined indicating ecology relating to sleep of the person to be examined or sleep ecology data of other persons other than the person to be examined. The measurement section measures the sleep duration of the person to be examined. The sleep sufficiency level estimation section determines an initial level of the sleep sufficiency levels from the maximum sleep duration and an ordinary sleep duration obtained from the sleep ecology data of the person to be examined or evaluation on the maximum sleep duration and the ordinary sleep duration by the person to be examined himself/herself, or data relating to the maximum sleep duration and the ordinary sleep duration obtained from other persons other than the person to be examined. The sleep sufficiency level estimation section further estimates the sleep sufficiency level of the person to be examined after sleep by calculating an increase/decrease of the sleep sufficiency level from the initial level of the sleep sufficiency level corresponding to the sleep duration of the person to be examined by using the function selected from the plurality of functions according to the sleep ecology data of the person to be examined obtained from the person to be examined and the sleep duration zone to which the initial level of the sleep sufficiency level of the person to be examined belongs.

### BRIEF DESCRIPTION OF THE DRAWINGS

FIG. 1 is a block diagram showing an embodiment of a configuration of a sleep sufficiency estimation system according to the present invention to which a method for estimating sleep sufficiency according to the present invention is carried out;

FIG. 2 is a flowchart of an embodiment of the method for estimating sleep sufficiency according to the present invention;

FIG. 3 is a graph showing an example of relationships between sleep sufficiency levels and sleep durations; and

FIG. 4 is a graph for explaining plural functions.

### DESCRIPTION OF EMBODIMENTS

Hereinafter, an embodiment of the present invention will be explained in detail with reference to the drawings. In a sleep sufficiency estimation system and a method for estimating sleep sufficiency according to the embodiment of the present invention to be described in detail below, a sleep sufficiency level of a person to be examined is estimated, and the person to be examined is notified of the result. The notification can be made with visualization or the like of the estimated result. Here, the sleep sufficiency level means a level indicating to what degree a sleep duration has been acquired with respect to the maximum value of a sleep duration necessary for the person to be examined. The sleep sufficiency level =the sleep duration/the maximum value of the sleep duration necessary for the person to be examined.

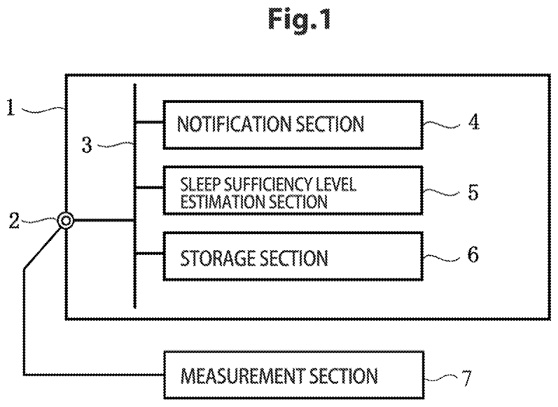

FIG. 1 is a block diagram showing a configuration of a sleep sufficiency estimation system 1 according to the embodiment of the present invention. As shown in FIG. 1, The sleep sufficiency estimation system 1 according to the embodiment of the present invention includes an input/output terminal 2, a bus 3 connected to the input/output terminal 2, a notification section 4, a sleep sufficiency level estimation section 5, and a storage section 6 respectively connected to the bus 3. Outputs from a measurement section 7 are inputted to the input/output terminal 2. The sleep sufficiency level estimation section 5 is realized by a computer. The storage section 6 can be also configured by a storage device such as a RAM included in the computer. The measurement section 7 is, for example, a well-known measuring instrument such as a wearable watch having a function of measuring an awakening duration or the sleep duration. When using the measuring instrument for measuring the awakening duration, a duration obtained by subtracting the awakening duration from 24 hours will be the sleep duration.

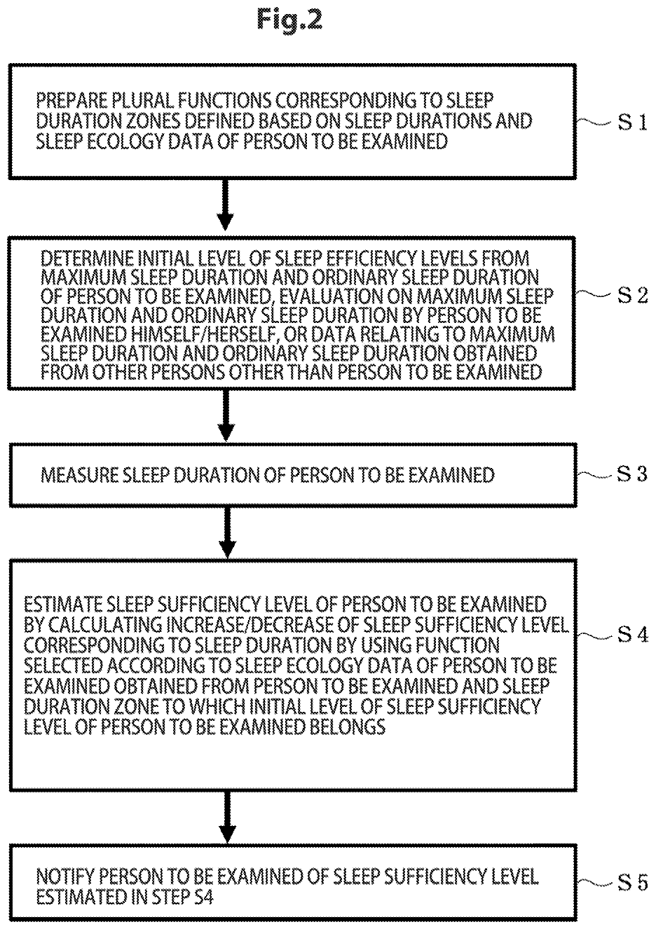

FIG. 2 is a flowchart showing the entire flow of a method including part of a flowchart of a program in a case where the method for estimating sleep sufficiency according to the embodiment of the present invention is executed by using a computer. A case where the method for estimating sleep sufficiency is realized by operation of the sleep sufficiency estimation system 1 shown in FIG. 1 will be explained as follows. However, it goes without saying that the method for estimating sleep sufficiency according to the present invention is not limited to the case of using the sleep sufficiency estimation system 1 and may be widely achieved by using hardware configured by combining a well-known computing device with a memory.

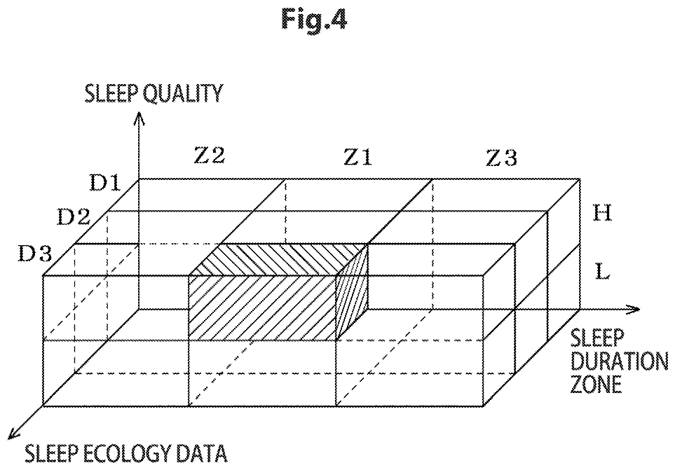

In Step S1 of the flowchart in FIG. 2, the storage section previously stores a plurality of plural functions corresponding to a plurality of sleep duration zones defined based on sleep durations and characteristics of the person to be examined (sleep ecology data). Here, as the plural functions corresponding to the plural sleep duration zones and the sleep ecology data of the person to be examined, an n-th order (“n” is a natural number) function in which the sleep duration (or the awakening duration) of the person to be examined is an independent variable and an increase/decrease of the sleep sufficiency level is a dependent variable is used. Examples of the functions will be explained later. The sleep ecology data of the person to be examined indicates characteristics of the person to be examined such as sex, age, sleep lifestyle habits, the maximum sleep duration, an ordinary sleep duration, and the presence of sleepiness in the daytime. In later-described FIG. 4, the sleep ecology data of persons to be examined is shown as D1 to D3. For example, the sleep ecology data of the person to be examined D1 to D3 may be defined by differences in sex and age in FIG. 4. For example, D1 to D3 can be defined as males in teens to twenties, males in thirties to fifties, and males in sixties or more.

Here, the storage section 6 may further store sleep ecology data (D1 to D3) that is characteristic data indicating characteristics of persons to be examined obtained from persons to be examined, other persons' data and/or research data concerning sleep ecology data obtained from persons other than the persons to be examined, and the plural functions corresponding to sleep qualities of the persons to be examined.

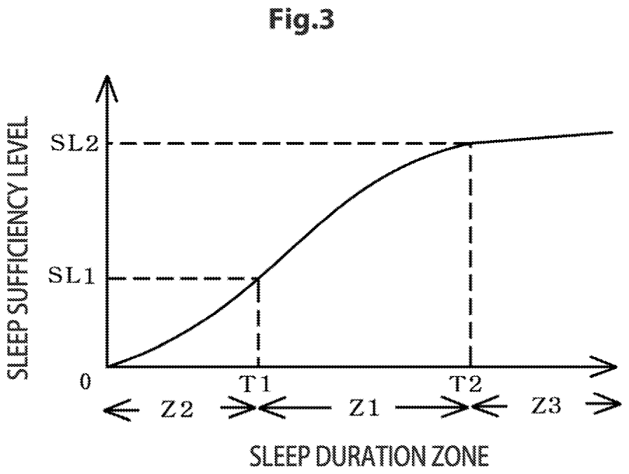

The above-described sleep duration zones and the plural functions will be explained with reference to FIG. 3. The sleep duration zones may be defined as any number of zones, and the simplest case will be explained as an example as follows. The plural sleep duration zones defined based on the sleep durations are zones of sleep durations defined based on the sleep sufficiency levels between one sleep sufficiency level and another sleep sufficiency level. In respective sleep duration zones, an increase/decrease rate of the sleep sufficiency level differs. For example, when the sleep duration zones are defined as three zones, three sleep duration zones can be defined as zones including (1) a first sleep duration zone Z1 in which the sleep sufficiency level in a state where sleep shortage and sleep longer than necessary do not occur can be obtained due to an adequate sleep duration, (2) a second sleep duration zone Z2 in which the increase/decrease rate of the sleep sufficiency level is smaller than the increase/decrease rate in the first sleep duration zone (Z1) due to a short sleep duration and sleep shortage, and (3) a third sleep duration zone Z3 in which the increase/decrease rate of the sleep sufficiency level is equal to or smaller than the increase/decrease rate in the first sleep duration zone (Z1) due to a long sleep duration and sleep longer than necessary.

An example of relationships between the sleep sufficiency levels and the sleep durations can be represented by a graph shown in FIG. 3. The graph shown in FIG. 3 is estimated from sleep ecology data of the person to be examined or sleep ecology data of other persons.

That is, when the sleep durations of the person to be examined are defined as, for example, the zone Z2 in which the sleep duration is “0 (zero)” or more and smaller than a time T1, the zone Z1 in which the sleep duration is the time T1 or more and smaller than a time T2, and the zone Z3 in which the sleep duration is the time T2 or more as shown in FIG. 3, it is considered that an increase speed (also called an accumulation speed) of the sleep sufficiency level in each sleep duration differs according to respective sleep duration zones as described below. In FIG. 3, the sleep sufficiency level is SL1 when the time duration is T1, and the sleep sufficiency level is SL2 when the time duration is T2. The ordinary sleep duration (the sleep duration at the time of starting estimation) for the person to be examined belongs to any of the sleep duration zones Z1 to Z3.

In FIG. 3, the sleep duration zone Z2 corresponds to a state where the sleep duration of the person to be examined is short, and the accumulation speed of the sleep sufficiency level corresponding to increase of the sleep duration is relatively slow in this zone as shown in FIG. 3. An average accumulation speed in the sleep duration zone Z2 can be obtained from a ratio of the sleep sufficiency level SL1 and the sleep duration T1. It can be considered that characteristics in the above sleep duration zone Z2 means that in a situation where the sleep duration is short, the person to be examined takes time to recover his/ her physical condition even if the sleep duration is increased.

In the sleep duration zone Z1, the person to be examined obtains an adequate sleep duration in daily life, in which the accumulation speed of the sleep sufficiency level is relatively higher as compared with other sleep duration zones Z2 and Z3 as shown in FIG. 3. This can be understood also from a fact that the average accumulation speed in the sleep duration zone Z1 obtained from a ratio of a difference between the sleep sufficiency level SL2 and the sleep sufficiency level SL1 and a difference between the sleep duration T2 and the sleep duration T1 has a larger value than average accumulation speeds in the other sleep duration zones Z2 and Z3. Characteristics in the above sleep duration zone Z1 means that it is possible to increase the sleep sufficiency level with less sleep duration as compared with the other sleep duration zones Z2 and Z3. Accordingly, it is most efficient to live within a range of the sleep duration zone Z1 in daily life for efficiency of utilizing living hours, efficiency of work, efficiency of effective restoring by sleep.

The sleep duration zone Z3 corresponds to a state where the person to be examined intends to increase the sleep efficiency level to a degree more than necessary in daily life, that is, intends to have sleep longer than necessary. As shown in FIG. 3, the accumulation speed of the sleep sufficiency level in the zone Z3 is in the same degree as or further slower than the accumulation speed in the sleep duration zone Z2. Characteristics in the above sleep duration zone Z3 means that considerable sleep duration is required for accumulating a sleep amount. It is desirable for the person to be examined to know about insufficiency in too short sleep duration or too long sleep duration and to change lifestyle habits so that an estimation result of the sleep efficiency level falls within the sleep duration zone Z1.

The sleep duration zones Z1 to Z3 have the above characteristics respectively. When the sleep duration (or 24 hours−awakening duration) is X, an increase/decrease in the sleep sufficiency level is Y, and the increase/decrease rate of the sleep sufficiency level (hereinafter referred to as merely a “coefficient”) per an unit sleep duration (or an unit awakening duration) in respective sleep duration zones Zn (n=1 to 3) is Kn (n=1 to 3), a relationship between X and Y can be represented by, for example, a linear function of the following formula (1).

Y=KnX (1)

In this case, a value of the coefficient Kn is a coefficient indicating the increase/decrease rate of the sleep sufficiency level per the unit sleep duration in the plural sleep duration zones Zn. The coefficient Kn is different for each of the sleep duration zones as described above. Here, the value of the coefficient Kn varies also due to the sleep ecology data of the person to be examined (D1 to D3 of FIG. 4) and/or high/low of sleep qualities (H or L of FIG. 4).

High/low in sleep qualities can be recognized based on characteristics in observed brain waves, and it is also possible to recognize the qualities by adopting methods of, for example, estimating the qualities from measurement values such as the number of tosses and turns, the number of vibrations of a pillow, a respiratory rate, heart rate variability, blood pressure variability and a perspiration amount by using the measurement instrument 7 such as the wearable watch.

The value of the coefficient Kn may be determined by being changed according to at least one of objective data taken from the person to be examined or other persons, subjective data taken from the person to be examined or other persons, and the increase/decrease rate of the sleep sufficiency level. Here, the objective data includes at least one of data indicating environment such as a season, temperature, body temperature, humidity, atmospheric pressure, an exercise amount, sunshine duration, weather, meal contents, a sound exposure amount, and a light exposure amount, an endocrine hormone amount, and menstrual rhythm. The subjective data or the objective data includes at least one of data indicating a physical condition or a mood. Therefore, it can be considered that the value of the coefficient Kn is adjusted, for example, so as to increase the value of the coefficient Kn in accordance with data indicating that the physical condition or the mood is good and so as to decrease the value of the coefficient Kn in accordance with data indicating that the physical condition or the mood is bad.

FIG. 4 shows a case where data D1 to D3 can be obtained as the sleep ecology data and the sleep quality is binarized into a state H where the sleep quality is high and a state L where the sleep quality is low as an example. When the data D1 to D 3 is about “sex and age”, D1 may be defined as males in teens to twenties, D2 may be defined as males in thirties to fifties, and D3 may be defined as males in sixties or more. The coefficient Kn in the sleep duration zones Z1 to Z3 takes different values in eighteen subspaces in a three-dimensional coordinate system shown in FIG. 4. For example, a subspace surrounded by oblique lines in FIG. 4 belongs to the data D3 as sleep ecology data and the state H where the sleep quality is high in the sleep duration zone Z1, and a value of the coefficient Kn corresponding to the subspace is set.

Accordingly, the storage section 6 stores the function formula (1) and the eighteen coefficient values to thereby previously store the eighteen functions in the above example.

Next, in Step S2, an initial level of the sleep sufficiency levels of the person to be examined is determined from the maximum sleep duration and the ordinary sleep duration of the person to be examined, or evaluation on the ordinary sleep duration of the person to be examined himself/herself. The initial level of the sleep sufficiency levels of the person to be examined may be determined from data obtained from other persons other than the person to be examined, that is, data of other persons or research data such as values calculated from big data. In Step S2, the initial level of the sleep sufficiency levels can be determined by calculating a ratio of the ordinary sleep duration measured by the person to be examined and the maximum sleep duration obtained by actual measurement or self-evaluation (an initial sleep duration/the maximum sleep duration), or by using evaluation on the ordinary sleep duration of the person to be examined himself/herself and so on. Here, the maximum sleep duration means a sleep duration necessary for the person to be examined, which can be obtained by actually measuring the sleep duration of the person to be examined in a state where the person to be examined can sleep without limitation or restriction. The evaluation on the ordinary sleep duration of the person to be examined himself/herself is obtained by determining the sleep duration necessary for himself/herself or the sleep sufficiency level at the moment by the person to be examined.

Next, in Step S3, the sleep duration of the person to be examined is measured. Specifically, activity amounts such as an exercise amount, posture and a body movement amount, and physiological information such as brain waves, a heart rate, blood pressure, skin potential, muscle potential, stomach sound, perspiration, and temperature are measured by using a well-known sensor such as the wearable watch. Moreover, to which state of sleeping or awakening the person to be examined belongs is determined by a well-known method. The sleep duration can be measured by using the above results. When the awakening duration is measured, a duration obtained by subtracting the awakening duration from 24 hours can be determined as the sleep duration. Here, when the sleep duration is measured, high/low in sleep quality in the person to be examined may be determined from the above measurement results.

Next, in Step S4, the increase/decrease of the sleep sufficiency level corresponding to the sleep duration is calculated by using characteristic data indicating characteristics of the person to be examined obtained from the person to be examined, namely, the sleep ecology data, and the function selected according to the sleep duration zone to which the initial level of the sleep sufficiency levels of the person to be examined belongs. Then, the sleep sufficiency level of the person to be examined is estimated from the calculation result (initial level ±increase/decrease). Here, the function may be selected further in consideration of the sleep quality.

In the method according to the present invention, the sleep sufficiency level of the person to be examined is determined based on the plural sleep duration zones defined based on the plural sleep durations and the function selected from plural functions corresponding to the sleep ecology data of the person to be examined: therefore, the sleep sufficiency level can be estimated at any time.

Specifically, for example, the sleep sufficiency level estimation section 5 selects the coefficient corresponding to the sleep duration zone to which the sleep state at the moment belongs, the sleep ecology data of the person to be examined, and the sleep quality from plural coefficients stored in the storage section 6 in Step S1 as described above, and calculates the increase/decrease of the sleep sufficiency level from the sleep duration measured in Step S3 using the formula (1).

In the embodiment, the sleep sufficiency level estimation section 5 receives the sleep ecology data, data indicating the sleep duration zone, and data concerning the sleep quality from the storage section 6 when performing the above calculation. These data may be inputted from the outside through the input/output terminal 2 and the bus 3.

In the case where the awakening duration is measured in Step S3, the sleep duration is calculated based on the awakening duration, and the increase/decrease of the sleep sufficiency level is calculated by using the coefficient previously stored in the storage section 6 selected according to the sleep duration zone and the sleep ecology data in the same manner as in the case where the sleep duration is measured.

As described above, the sleep sufficiency level estimation section 5 can estimate the latest sleep sufficiency level of the person to be examined by adding the increase/decrease to the initial level of the sleep sufficiency level to thereby update the sleep sufficiency level.

Then, in Step S5, the sleep sufficiency level estimated in Step S4 is notified to the person to be examined. Specifically, for example, the notification section 4 notifies the person to be examined of the sleep sufficiency level by displaying the latest sleep sufficiency level estimated by the sleep sufficiency level estimation section 5 on a monitor, or by adopting sensuous methods such as making a sound.

Note that the function shown in the formula (1) may be generally an n-th order (n is a natural number) function, and the coefficient of the n-th order function can be dealt with in the same manner as the coefficient Kn.

As described above, the sleep sufficiency level of the person to be examined can be estimated at any time, and further, the results thereof can be notified to the person to be examined when adopting the sleep sufficiency estimation system and the method for estimating the sleep sufficiency according to the embodiment of the present invention.

What is claimed is:

1. A method for estimating sleep efficiency comprising:
a first step of preparing a plurality of functions indicating relationships between a plurality of sleep sufficiency levels and a plurality of sleep duration zones defined based on a plurality of sleep durations in consideration of sleep ecology data of a person to be examined indicating ecology relating to sleep of the person to be examined or sleep ecology data of other persons other than the person to be examined;
a second step of determining an initial level of the sleep sufficiency levels from the maximum sleep duration and an ordinary sleep duration obtained from the sleep ecology data of the person to be examined or evaluation on the maximum sleep duration and the ordinary sleep duration by the person to be examined himself/herself, or data relating to the maximum sleep duration and the ordinary sleep duration obtained from other persons other than the person to be examined;
a third step of measuring the sleep duration of the person to be examined; and
a fourth step of estimating the sleep sufficiency level of the person to be examined after sleep by calculating an increase/decrease of the sleep sufficiency level from the initial level of the sleep sufficiency level corresponding to the sleep duration of the person to be examined by using the function selected from the plurality of functions according to the sleep ecology data of the person to be examined obtained from the person to be examined and the sleep duration zone to which the initial level of the sleep sufficiency level of the person to be examined belongs.

2. The method for estimating sleep efficiency according to claim 1,
wherein, in the first step, an n-th order function with n being a natural number, in which the sleep duration is an independent variable and the increase/decrease of the sleep sufficiency level is a dependent variable, is used as the function.

3. The method for estimating sleep efficiency according to claim 2,
wherein a coefficient of the n-th order function is determined according to at least one of objective data taken from the person to be examined or other persons, subjective data taken from the person to be examined or other persons, and an increase/decrease rate of the sleep sufficiency level already estimated.

4. The method for estimating sleep efficiency according to claim 3,
wherein the function is represented by a linear function of Y=KnX, where X is the sleep duration, Y is an increase/decrease of the sleep sufficiency level, and Kn is a coefficient indicating the increase/decrease rate of the sleep sufficiency level per an section sleep duration in the plurality of sleep duration zones Zn.

5. The method for estimating sleep efficiency according to claim 1,
wherein, in the first step, the plurality of functions are further prepared in consideration of sleep qualities different according to the sleep ecology of the person to be examined, and
in the fourth step, an appropriate function is further selected from the functions prepared in consideration of the sleep quality.

6. The method for estimating sleep efficiency according to claim 4,
wherein, in the first step, the plurality of sleep duration zones include a first sleep duration zone in which the sleep sufficiency level in a state where sleep shortage and sleep longer than necessary do not occur is capable of being obtained due to an adequate sleep duration, a second sleep duration zone in which the increase/decrease rate of the sleep sufficiency level is smaller than the increase/decrease rate in the first sleep sufficiency zone due to a short sleep duration and sleep shortage, and a third sleep duration zone in which the increase/decrease rate of the sleep sufficiency level is equal to or smaller than the increase/decrease rate in the first sleep sufficiency zone due to a long sleep duration, namely, due to sleep longer than necessary.

7. The method for estimating sleep efficiency according to claim 1, further comprising:
a fifth step of notifying the person to be examined of the sleep sufficiency level estimated by the fourth step.

8. The method for estimating sleep efficiency according to claim 3,
wherein the objective data includes at least one of data indicating a season, temperature, body temperature, humidity, atmospheric pressure, an exercise amount, sunshine duration, weather, meal contents, a sound exposure amount, a light exposure amount, an endocrine hormone amount, and menstrual rhythm, and
the subjective data includes at least one of data indicating a physical condition or a mood.

9. A sleep sufficiency estimation system, comprising:
a storage section storing a plurality of functions indicating relationships between a plurality of sleep sufficiency levels and a plurality of sleep duration zones defined based on sleep durations in consideration of sleep ecology data of a person to be examined indicating ecology relating to sleep of the person to be examined or sleep ecology data of other persons other than the person to be examined;
a measurement section measuring the sleep duration of the person to be examined; and
a sleep sufficiency level estimation section determining an initial level of the sleep sufficiency levels of the person to be examined from the maximum sleep duration and an ordinary sleep duration obtained from the sleep ecology data of the person to be examined or evaluation on the maximum sleep duration and the ordinary sleep duration by the person to be examined himself/herself, or data relating to the maximum sleep duration and the ordinary sleep duration obtained from other persons other than the person to be examined, and estimating the sleep sufficiency level of the person to be examined after sleep by calculating an increase/decrease of the sleep sufficiency level from the initial level of the sleep sufficiency level corresponding to the sleep duration of the person to be examined by using the function selected from the plurality of functions according to the sleep ecology data of the person to be examined obtained from the person to be examined and the sleep duration zone to which the initial level of the sleep sufficiency level of the person to be examined belongs.

10. The sleep sufficiency estimation system according to claim 9,
wherein the storage section further stores the plurality of functions prepared in consideration of sleep qualities different according to the sleep ecology of the person to be examined, and
the sleep sufficiency level estimation section further selects an appropriate function in consideration of the sleep quality.

11. The sleep sufficiency estimation system according to claim 9,
wherein the plurality of sleep duration zones include a first sleep duration zone in which the sleep sufficiency level in a state where sleep shortage and sleep longer than necessary do not occur is capable of being obtained due to an adequate sleep duration, a second sleep duration zone in which the increase/decrease rate of the sleep sufficiency level is smaller than the increase/decrease rate in the first sleep sufficiency zone due to a short sleep duration and sleep shortage, and a third sleep duration zone in which the increase/decrease rate of the sleep sufficiency level is equal to or smaller than the increase/decrease rate in the first sleep sufficiency zone due to a long sleep duration, namely, due to sleep longer than necessary.

12. The sleep sufficiency estimation system according to claim 9, further comprising:
a notification section notifying the person to be examined of the sleep sufficiency level estimated by the sleep sufficiency level estimation section.

13. The sleep sufficiency estimation system according to claim 9,
wherein the function is an n-th order function with n being a natural number, in which the sleep duration of the person to be examined is an independent variable and the increase/decrease of the sleep sufficiency level is a dependent variable.

14. The sleep sufficiency estimation system according to claim 13,
wherein a coefficient of the n-th order function is determined according to at least one of objective data taken from the person to be examined or other persons, subjective data taken from the person to be examined or other persons, and an increase/decrease rate of the sleep sufficiency level already estimated.

15. The sleep sufficiency estimation system according to claim 14,
wherein the objective data includes at least one of data indicating a season, temperature, body temperature, humidity, atmospheric pressure, an exercise amount, sunshine duration, weather, meal contents, a sound exposure amount, a light exposure amount, an endocrine hormone amount, and menstrual rhythm, and
the subjective data includes at least one of data indicating a physical condition or a mood.
